(19) United States
(12) Patent Application Publication
(10) Pub. No.: US 2012/0086810 A1
(43) Pub. Date: Apr. 12, 2012

(54) METHOD FOR A TOUCHLESS DETERMINATION OF THE TEMPERATURE OF AN OBJECT AND CORRESPONDING THERMAL IMAGING CAMERA

(75) Inventor: Andreas Messerschmid, Bonndorf-Gundelwangen (DE)

(73) Assignee: TESTO AG, Lenzkirch (DE)

(21) Appl. No.: 13/268,013

(22) Filed: Oct. 7, 2011

(30) Foreign Application Priority Data
Oct. 9, 2010 (DE) 10 2010 048 022.3

(51) Int. Cl. H04N 5/33

(52) U.S. Cl. 348/164; 348/E05.09

(57) ABSTRACT

A method for a touchless determination of the temperature of an object (13) in which at least one IR-image (10) and at least one VIS-image (12) of the object (13) are generated with an IR-detector unit (2) and with a VIS-detector unit (3) of a thermal imaging camera (1), a feature detection (16) is applied to the IR-image (10) and/or the VIS-image (12), thus extracting features (17), and identifying the object (13) from the features (17) extracted and present as a result of the feature detection (16), and to determine and display from the IR-image (10) at least one temperature value (25) of the object (13).

### CROSS-REFERENCE TO RELATED APPLICATIONS

This application claims the benefit of German Patent Application No. DE 10 2010 048 022.3, filed Oct. 9, 2010, which is incorporated herein by reference as if fully set forth.

### BACKGROUND

The invention relates to a method for the touchless determination of the temperature of an object.

The invention further relates to a thermal imaging camera with an IR-detector unit, which is provided to record an IR-image, with a VIS-detector unit, which is provided to record a VIS-image, with a data processing unit and an output unit connected to the data processing unit.

Thermal imaging cameras, which allow the recording of IR-images reflecting thermal information, have proven useful in many applications.

For example, thermal imaging cameras are used in order to find persons showing an elevated body temperature in airports or other places with a high volume of people. This occurs by the operating personnel pointing a thermal imaging camera to the persons to be checked, thus allowing a measure of the body temperature of this person. The operating personnel of the camera are here responsible in detail, that the camera is correctly aligned and focused and that the temperature is read correctly and subsequently assessed, if for example due to an elevated body temperature a feverish disease might be present.

### SUMMARY

The invention is based on the objective to simplify such methods.

For this purpose, the invention provides the following steps in the method mentioned at the outset:
in a first step, at least one IR-image and at least one VIS-image of the object are generated,
in a second step at least one IR-image and at least one VIS-image are fed to a feature detection device,
in a third step the feature detection device performs at least one feature detection in the images supplied,
in a fourth step, using the result of at least one feature detection, the object is identified in an object identification and
in a fifth step, using at least one IR-image, at least one temperature measurement of the identified object is deduced and displayed.

As a result of the feature detection preferably one feature or several features is/are extracted by digital image processing.

The invention therefore allows an automated and touchless detection and determination of temperatures of predefined objects. Here, the invention uses the knowledge that, although neither the IR-channel recording the IR-image nor the visual image channel recording the VIS-image produce a flawless detection of objects or features for each recorded scene, however, by a suitable combination of the information from the two image channels the quality of the object recognition can be decisively improved.

It has shown that in the IR-channel (in the infrared spectral range of light, IR) the quality of the object recognition depends on the contrast given and thus on sufficient temperature differences present, while in the visual image channel (in the visual spectral range of light, VIS) the quality of the object recognition depends on sufficient contrast, which is ensured by differences in color and/or brightness of the VIS-image.

Thus, the invention can be used for objects showing no temperature difference or only a slight temperature difference in reference to the background and cannot or can only with difficulty be detected in the IR-channel.

According to the invention, in this case additional information from the visual image channel can be used. The invention is also applicable advantageously for scenes that were recorded in the visual image channel at total or almost total darkness or identical coloring of the object in reference to the background. In this case, information from the IR-channel can be used for object identification.

The invention can advantageously be used in a wide range of applications. Depending on application, different tables of features may be predetermined to recognize features in order to identify the desired objects.

For example, in the application mentioned at the outset predetermined typical facial features of a person may be provided. This may further occur, for example, in production processes by presetting characteristic shapes, colors, surface structures, and the like of the objects to be monitored, such as products to be produced. In general, feature descriptions from digital image processing can be used for recognizing features.

Here it is beneficial if a realistic image position is stored in reference to the identified object, from which the temperature value is deduced in the IR-image.

The deduction of the temperature value from the IR-image occurs here in a manner known per se.

Here, it may be provided that the deduced temperature value is referenced to a temperature threshold stored and that a control signal is generated from the result of said comparison, for example an alarm. In an embodiment according to the invention it may be provided that the IR-image and at least one VIS-image are correlated in a second step before feature recognition is performed.

For this purpose, it may be provided that at least one IR-image and at least one VIS-image are combined in a second step of the feature recognition device, by a joint image being calculated from at least one IR-image and from at least one VIS-image, with said joint image including image information both from the IR-image as well as the VIS-image. At least one feature recognition is performed in the third step based on the joint image.

For example it may be provided that only the brightness information of the VIS-image is interfered with the IR-image. Here, it is advantageous that this way the contrast in the IR-image is increased and thus the detection of features is facilitated.

However, it may also be provided that in the third step in the feature recognition device a first feature recognition is performed at least at an IR-image and a second feature recognition at least at one VIS-image, with a joint data field being deduced from the respective results of the feature recognition, which in the fourth step is processed to identify the object as the result of at least one feature recognition. Here it is advantageous that the first and/or the second feature recognition can each be applied in case of an IR-image and/or a VIS-image, which may lead to an improvement of the feature recognition. In a two-dimensional arrangement the data field comprises numeric values resulting from a pixel-wise combination or compensation of both images.

The joint data field can here be deduced by way of pixel-wise addition, subtraction, and/or multiplication of values from a data field of the result of the first feature recognition with a data field of the result of the second feature recognition. This way, a joint data field can be created in an easily implemented fashion for further processing.

In order to realize the temperature measurement of mobile objects, the so-called “object tracking”, it may be provided that at least one IR-image is an element of an IR-image sequence of IR-images and that the object identification and/or the feature recognition is/are controlled such that an object is identified in the IR-image, which was previously identified in an IR-image preceding the IR-image sequence.

Here, the VIS-image may also include a VIS-image sequence. In this case, the VIS-image sequence may be evaluated simultaneously, for example, in reference to the IR-image sequence or individual images may be allocated respectively for evaluation.

In one embodiment it may be provided that the joint image is calculated in the second step as a mixed image with a constant mixing factor. Thus, a so-called “alpha blending” is realized. The mixing factor may also vary over the image depending on the image position.

According to the invention, a multitude of different algorithms may be used to detect features.

Preferably it is provided that in the third step the feature recognition is realized via object identification and in the fourth step as “Harris-Corner-Detector”. Other image processing algorithms may also be used advantageously, such as “Hough-Transformation”.

According to an embodiment of the invention it may be provided that in the third step first a feature recognition is performed at the supplied IR-image, that subsequently the result of the feature recognition is checked for usefulness for object identification by deducting a usefulness value from the result, and that upon a usefulness threshold being fallen short a second feature recognition is performed at the supplied VIS-image, with the result being processed in the second feature recognition or both feature recognitions are processed in the fourth step as the result of said feature recognition.

Here, it is advantageous that this way a self-adapting method is provided, in which the visual image channel, i.e. the VIS-image, is only evaluated when the feature recognition for the IR-image failed to yield satisfactory results. This way, computing time can be saved.

Here, particularly good results can be achieved when in the third step the second feature recognition is controlled depending on the deduced usefulness value. This may occur, for example by predetermining parameters.

In any case, it has proven beneficial when a calibration is performed to correlate the IR-images with the VIS-images, on the one hand compensating the off-set of the two optical axes of the image recording systems—i.e. an IR-detector unit on the one side and a VIS-detector unit on the other side, perhaps comprising corresponding optics—as well as the distortions of the respective optics.

This way it is easily possible to correlate spatial information concerning an object reliably identified in the VIS-image with the IR-image and thus to yield the position of the object of interest in the IR-image.

In order to attain the above-mentioned objective it is provided in a thermal imaging camera of the type mentioned at the outset that a feature recognition device can be realized with the data processing device and that the data processing device is embodied to perform a method according to the invention. This way, a compact device is created, which realizes the method according to the invention in a simple fashion. Here, the implementation of the data processing device can occur by way of suitable programming, however, individual or all processing steps may also be realized by a fixed electronic circuitry of discrete elements.

### BRIEF DESCRIPTION OF THE DRAWINGS

The invention is now explained in greater detail using an exemplary embodiment, however, it is not limited to the exemplary embodiment. Additional exemplary embodiments result from the combination of one or more features of the exemplary embodiment with each other and/or with one or more features of the claims.

Shown are:

FIG. 1: a thermal imaging camera according to the invention in a front view,

FIG. 2: a thermal imaging camera according to the invention in a rear view, and

FIG. 3: a schematic flow chart of the method according to the invention.

### DETAILED DESCRIPTION OF THE PREFERRED EMBODIMENTS

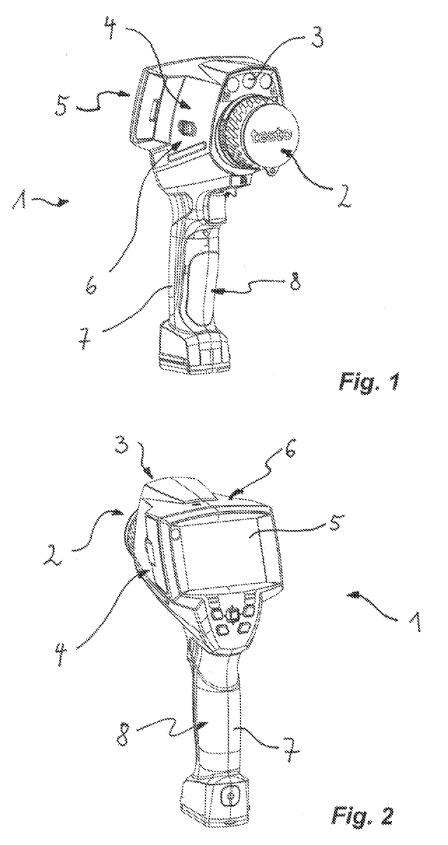

FIG. 1 shows a thermal imaging camera, marked 1 in its entirety in a three-dimensional diagonal view from the front. FIG. 2 shows the thermal imaging camera 1 in a three-dimensional diagonal view from the rear.

The thermal imaging camera 1 comprises in a manner known per se an IR-detector unit 2 with a sensor field of bolometers, not shown in greater detail, and an upstream arranged IR-optic, which defines the optic axis for recording IR-images.

The thermal imaging camera 1 further comprises a VIS-detector unit 3, which is embodied and set as a digital camera with an optic for recording VIS-images. This optic also defines an optic axis.

Inside the thermal imaging camera 1, a data processing device 4 is arranged, not shown in greater detail. In order to put out or display recorded IR-images and/or VIS-images and/or processed images deducted therefrom, an output unit 5 is provided in the form of a display at the back of the thermal imaging camera 1.

The thermal imaging camera 1 may comprise a data transmission unit as another output unit for a touchless and/or wired data communication with an external data processing device, for example a PC or the like.

The data processing device 4 of the thermal imaging camera 1 is arranged by programming a programmable logic component and/or by an electric circuitry of discrete parts such that the method described in the following can be implemented in the thermal imaging camera 1.

In particular, the data processing device 4 realizes a feature detection device 6 by its circuitry/programming.

The thermal imaging camera 1 receives the energy required for operation from an energy storage medium 8, a battery, or the like arranged in its handle 7.

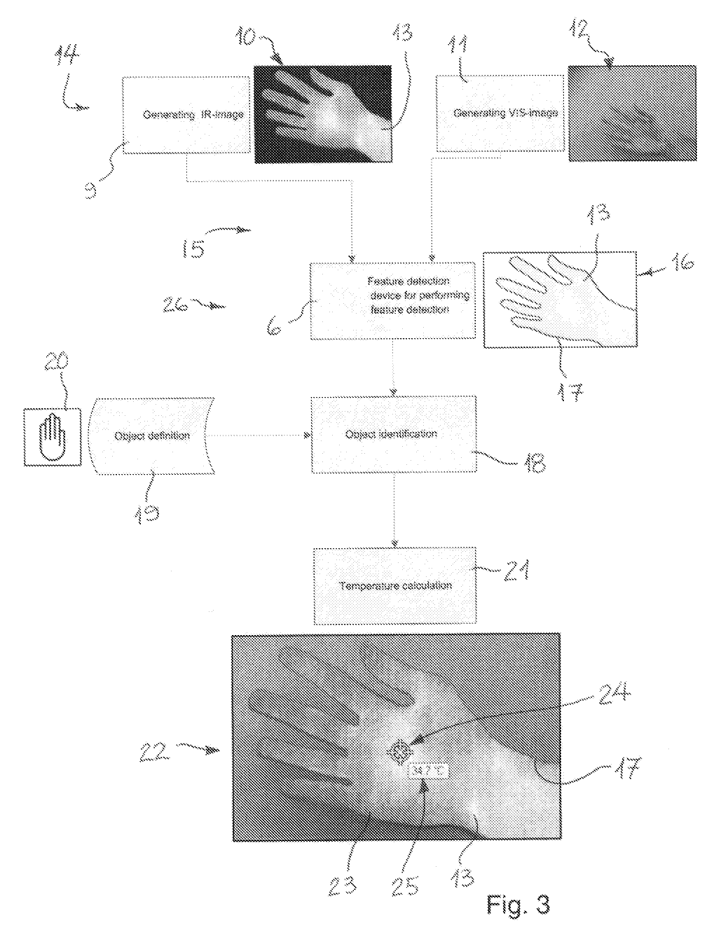

According to the invention, in the thermal imaging camera 1 the method is performed schematically shown in FIG. 3.

In a first step, initially the image generation 9 of an IR-image 10 occurs. This occurs via the IR-detector unit 2. Simultaneously or almost simultaneously the image generation 11 of a VIS-image 12 occurs via the VIS-detector unit 3.

By aligning the optic axes of the IR-detector unit 2 and the VIS-detector unit 3 as well as the timely image generations 9, 11 the IR-image 10 and the VIS-image 12 show different representations of the same object 13, for example a human hand.

Thus, image generation 9 and image generation 11 form the first step 14 of the method according to the invention.

In a second step 15 at least one IR-image 10 as well as at least one VIS-image 12 are fed to a feature recognition device 6.

Here, this feeding may occur such that first a joint image is calculated from at least one IR-image 10 and from at least one VIS-image 12. This joint image then includes image information both from the IR-image 10 as well as the VIS-image 12. For example, the temperature information in the form of a color portion of the IR-image 10 and the contrast information in the form of the brightness portion of the VIS-image 12 may be combined in a joint image.

Alternatively it is provided that the IR-image 10 and the VIS-image 12 of the feature detection device 6 are supplied separately and here processed separately.

At least one feature detection 16 is performed in the feature detection device. At least one predetermined feature is extracted from the feature detection.

In FIG. 3 the result of such feature detection 16 is shown as an example, thus the extracted feature, shown as the contour line 17 of the hand 13. Using the feature detection 16, different features, known from digital image processing as well as multi-dimensional features, for example feature descriptors, may be used as well, which in the respectively concrete application are better suited.

In a fourth step 18, an object identification, an object 13 is identified using the result of at least one feature detection 16.

This occurs by accessing the object definition 19 stored in the thermal imaging camera 1, in which tables of features 20 are stored and provided for feature comparison.

In a fifth step 21, finally a processed image 22 is created and/or displayed via the display unit 5.

The processed image 22 includes a representation of the object 13, which was created from the illustration of the IR-image 10 by adding the image data of the VIS-image 12.

A processed image 22 is shown schematically in FIG. 3, as an example and sufficient for many practical applications, which was composed from the contour line 17, extracted from the VIS-image 12, and from the falsified colored representation 23 of the IR-image 10.

The thermal imaging camera 1 may also be embodied to display other mixed versions or interferences of the images 10, 12.

With regards to the extracted and identified contour line 17 or generally the extracted feature a relative position is saved in the thermal imaging camera 1, by which the respectively desired measuring spot 24 is defined.

This way, by processing the IR-image 10 a temperature value 25 can be deduced for the measurement spot 24 from the processed image 22 or directly from the IR-image 10.

This temperature value 25 is displayed in FIG. 3, for example by showing the numeric value in the processed image 22.

Other ways of displaying temperature values may also be implemented.

In the feature detection device 6 different feature recognitions may be performed for each the IR-image 10 and the VIS-image 12. Here, it may be provided that the result of the feature detection 16 for the IR-image 10 is subsequently checked for the question if the performance of a feature detection is even necessary for the VIS-image 12, or if the results already provided are sufficient for a reliable further execution of the method.

In case of a separate feature detection 16, the results are recorded in a joint data field by adding, subtracting, and/or multiplying the individual results pixel-wise.

The final result yielded this way is now further processed in object identification in the fourth step 18.

Alternatively it may be provided that prior to the feature detection 16 a joint image is created by overlapping and/or interfering the IR-image 10 and the VIS-image 12.

In order to combine the IR-image 10 and the VIS-image 12 before or after the feature detection 16 here correlating data from a calibration is stored in the thermal imaging camera 1, by which an off-set of the optic axes of the IR-detector unit 2 and the VIS-detector unit 3 and/or imaging errors of the respective optics can be compensated in reference to each other.

In a step, not shown in greater detail in FIG. 3, it is provided that the deduced temperature value 25 is further processed in a monitoring device, with a control signal, for example an alarm, being generated according to settings, when a threshold is exceeded and/or falls short.

The thermal imaging camera 1 can create IR image sequences from IR-images 10 and VIS-image sequences from VIS-images 12 in a continuous operation.

When the object identification is controlled in the fourth step 18 such that an object 13 is identified in the respectively processed IR-image 10, which previously was already identified in the IR-image 10 of the IR-image sequence, an “Object-Tracking”and/or a detection of the temporal change of the object 13 can be realized.

In the method for a touchless determination of the temperature of an object 13 it is provided to generate at least one IR-image 10 and at least one VIS-image 12 of the object 13 using an IR-detector unit 2 and a VIS-detector unit 3 of a thermal imaging camera 1, applying a feature detection 16 for the IR-image 10 and/or the VIS-image 12, and here extract features 17, identifying the object 13 from the extracted features 17 present as the result of the feature detection 16, and deducing and displaying at least one temperature value 25 of the object 13 from the IR-image 10.

1. A method for touchless determination of a temperature of an object (13), comprising:
in a first step (9, 11, 14) generating at least one IR-image (10) and at least one VIS-image (12) of the object (13),
in a second step (15) feeding the at least one IR-image (10) and the at least one VIS-image (12) to a feature detection device (6),
in a third step (26) performing at least one feature detection (16) on the images supplied with the feature detection device (6),
in a fourth step (18), using the result (17) of at least one feature detection (16), identifying the object (13) in an object identification, and
in a fifth step (21), using the at least one IR-image (10), determining and displaying at least one temperature value (25) of the identified object (13).

2. The method according to claim 1, wherein the at least one IR-image (10) and the at least one VIS-image (12) are fed to the feature recognition device (6) in the second step (15), by a joint image being calculated from the at least one IR-image (10) and from the at least one VIS-image (12), which includes joint image information from both the IR-image (10) as well as the VIS-image (12), with the at least one feature detection (16) being performed on the joint image in the third step (26).

3. The method according to claim 1, wherein in the third step (26) a first feature detection (16) is performed at least on one of the IR-images (10) in the feature detection device (6) and a second feature detection (16) is performed at least on one of the VIS-images (12), with a joint data field being deduced pixel-wise from respective results (17) of the feature detections (16), which in the fourth step (18) are processed for identifying the object (13) as a result of the at least one feature detection (16).

4. The method according to claim 3, wherein the joint data field is deduced pixel-wise by adding, subtracting, or multiplying values of a data field of the result (17) of the first feature detection (16) with a data field of the result of the second feature detection (16).

5. The method according to claim 4, wherein the at least one IR-image (10) is an element of an IR-image sequence of IR-images (10) and the object identification (18) is controlled such that an object (13) is identified in the IR-image (10), which has previously been identified in the IR-image (10) of a previous IR-image sequence.

6. The method according to claim 2, wherein in the second step (15) the joint image is calculated with a constant or an image-position dependent mixing factor.

7. The method according to claim 1, wherein the at least one feature recognition (16) in the third step (26) utilizes a Harris-Corner-detector for object identification in the fourth step (18).

8. The method according to claim 1, wherein in the third step (26) first a feature recognition (16) is performed in the IR-image (10) supplied, and subsequently the result (17) of the feature recognition (16) is checked for suitability for object identification (18) by deducing a suitability value from the result (17), and in case the suitability values falls short of a suitability threshold, a second feature detection (16) is performed in the VIS-image (12) supplied, and the result (17) of the second feature detection (16) or both feature detections (16) is processed in the fourth step (18) as the result (17) of the feature detection (16).

9. The method according to claim 1, wherein in the third step (26), the second feature detection (16) is controlled depending on a deduced suitability value.

10. A thermal imaging camera (1) with an IR-detector unit (2), which is equipped to record an IR-image (10), with a VIS-detector unit (3), which is equipped to record a VIS-image (12), with a data processing device (4) and a display unit (5), which is connected to the data processing unit (4), wherein a feature detection device (6) is realized with the data processing device (4) and the data processing device (4) is equipped to perform a method according to claim 1.
